(19) United States
(12) Patent Application Publication
(10) Pub. No.: US 2018/0292422 A1
(43) Pub. Date: Oct. 11, 2018

(54) Apparatus for representing breath alcohol concentration with qualitative sensory output integrated in decorative ornamentation.

(71) Applicants: Andrew George Harper, West Sacramento, CA (US); Angielee DiNinni, West Sacramento, CA (US)

(72) Inventors: Andrew George Harper, West Sacramento, CA (US); Angielee DiNinni, West Sacramento, CA (US)

(21) Appl. No.: 15/946,684

(22) Filed: Apr. 5, 2018

Related U.S. Application Data
(60) Provisional application No. 62/482,187, filed on Apr. 6, 2017.

(51) Int. Cl. G01N 33/98; G01N 33/00

(52) U.S. Cl. CPC G01N 33/98; G01N 33/0009

(57) ABSTRACT

A breath alcohol concentration measurement system for use in monitoring the breath alcohol concentration of individuals, other than the primary owner, comprising, measuring breath alcohol concentration with an alcohol sensor, sending the data to a processor and representing the data qualitatively with a means of visual, auditory, or tactile sensory output that is integrated with a decorative ornamentation.

### CROSS REFERENCE TO RELATED PATENTS

This patent application claims the benefits of U.S. Provisional Patent Application No. 62/482,187 filed on Apr. 6, 2017 and makes reference to U.S. Pat. No. 8,590,363, U.S. Pat. No. 7,797,982, U.S. Pat. No. 8,418,796, U.S. Pat. No. 9,260,012.

### FIELD OF INVENTION

The technology relates to the general field of consumer electronics, and has certain specific application to chemical sensors and decorative electronics.

### BACKGROUND AND RELATED ART

Breath alcohol measuring systems, known as breathalyzers, operate on the principle that the alcohol content present in a subject's breath is proportional to their blood alcohol content (B.A.C.). Such a device is a non-invasive way to estimate B.A.C. Estimating B.A.C. is important because each state has laws that prohibit drivers with a certain level of B.A.C. from operating motor vehicles. Indeed, many states prohibit drivers from operating vehicles with any level of B.A.C. above zero.

There are a variety of breathalyzers available on the market. Police officers are commonly equipped with sophisticated handheld breathalyzer devices that can accurately measure the B.A.C. of suspected drunk drivers. Consumers also can buy a range of commercially available breathalyzer devices for personal use, as seen in U.S. Pat. No. 8,590,363. These personal breathalyzers are small and can be easily carried in a purse, pocket, or even on a key ring. Most of these handheld breathalyzers utilize a digital display to show the user his or her estimated B.A.C. to a certain degree of accuracy, as in seen in U.S. Pat. No. 7,797,982 to Burke.

An owner of a personal breathalyzer device can use it to make informed decisions about whether he or she, or some third party, can lawfully drive. This can be particularly useful for hosts of social gatherings. The consumption of alcohol is commonplace at many religious, social, sporting, or other celebratory gatherings. And in some states, hosts of social gatherings can face liability if their guests become intoxicated and then drive and injure third parties.

Although personal breathalyzer devices are well-adapted for self-monitoring B.A.C., I have found that it can be difficult to suggest to a third party that he or she ought to use the breathalyzer before driving. Asking someone else to use a breathalyzer is often viewed as an affront to his or her autonomy or personal decision making. Many people find it insulting to be asked to blow into a breathalyzer before driving. Due in part to the embarrassment that may be caused by using a breathalyzer in public, prior art has aimed to disguise the breathalyzer itself. Such prior art includes: U.S. Pat. No. 8,418,796 to Flores and U.S. Pat. No. 9,260,012 to Lopez, which both disguise the breathalyzer ignition interlock system. But while this prior art can alleviate the embarrassment associated with breathalyzing oneself in public, it does not mitigate the social stigma associated with attempting to breathalyze someone else.

It thus would be useful to have a way of testing the B.A.C. of third parties at social gatherings while minimizing the possibility of confrontation. The present invention satisfies this need and others.

### SUMMARY

This invention comprises a breathalyzer, a means of sensory output (such as lights, a speaker, a noise maker, or a vibrational apparatus), and a decoration with which the sensory output is integrated. The user blows a breath sample into the breathalyzer and the decoration qualitatively represents the user's breath alcohol concentration. The system creates a light-hearted atmosphere that helps alleviate the social stigma associated with asking another individual to use a traditional breathalyzer.

In an embodiment of the invention, the handheld wireless breathalyzer transmitter has a mouth piece with an alcohol sensor capable of accepting breath of a user. The user breathes into the mouthpiece and the alcohol sensor generates a signal received by a processor. The processor transmits the alcohol-concentration data through a wireless transmitter circuit to a base unit, which controls an array of holiday string lights that are wrapped around a Christmas tree in an ascending fashion—the first strand wrapped around the base of the tree and the last strand wrapped around the top of the tree. The higher the alcohol concentration sensed by the transmitter, the greater the number of string lights that will be illuminated. If the transmitter senses no alcohol, none of the lights will be illuminated. This embodiment provides a light-hearted, inoffensive way of testing for alcohol on the breath of a guest, avoiding confrontational stigma that a traditional breathalyzer may have.

### BRIEF DESCRIPTION OF THE DRAWINGS

FIG. 1 is an isometric view of the wireless breathalyzer transmitting unit of a preferred embodiment.

FIG. 2 is side view of the wireless breathalyzer transmitting unit of a preferred embodiment showing the power switch.

FIG. 3 is an electrical schematic for the wireless breathalyzer transmitting unit depicted in FIGS. 1 and 2.

FIG. 4 is a top view of the receiving unit of the same preferred embodiment shown in FIGS. 1 and 2.

FIG. 5 is an electrical schematic for the receiving unit of a preferred embodiment.

FIG. 6 a is an illustration demonstrating the use and functionality of a preferred embodiment shown in FIGS. 1, 2, 3, 4 and 5.

FIG. 7 shows the increasing illumination of the tree shown in FIG. 6.

FIG. 8 shows a second embodiment in which the breath alcohol concentration sensed by the breathalyzer transmitting unit is proportional to the number of light emitting diodes (LEDs) that are not illuminated in a clockwise fashion around the board.

FIG. 9 shows the back side of this dart board where the battery powered receiving unit that controls the LEDs are housed. Also shown is a protruding hang point to allow the board to be hung flush against a surface.

FIG. 10 shows a third embodiment in which the breath alcohol sensor, processor and sensory output are all integrated into one decoration, in this case a sports sign. The sensory output in this embodiment consists of an array of LEDs but also a small speaker. The processor illuminates and array of LEDs located on the initials of the sports team sign while also playing a sweeping tone on the speaker.

FIG. 11 shows the rear side of the sign with a cut out to house the electronics.

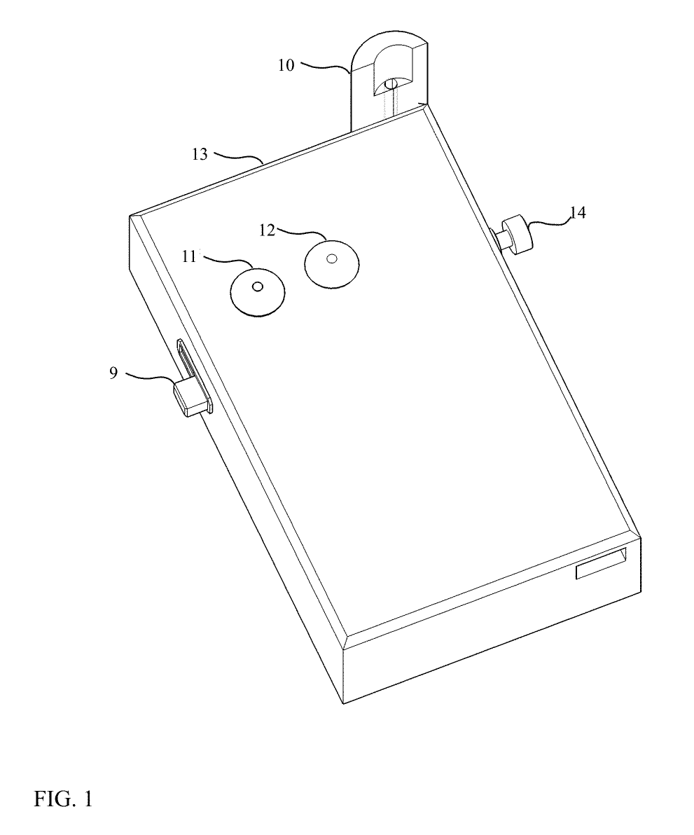

### DETAILED DESCRIPTION—FIGS. 1-5—FIRST EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalents; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

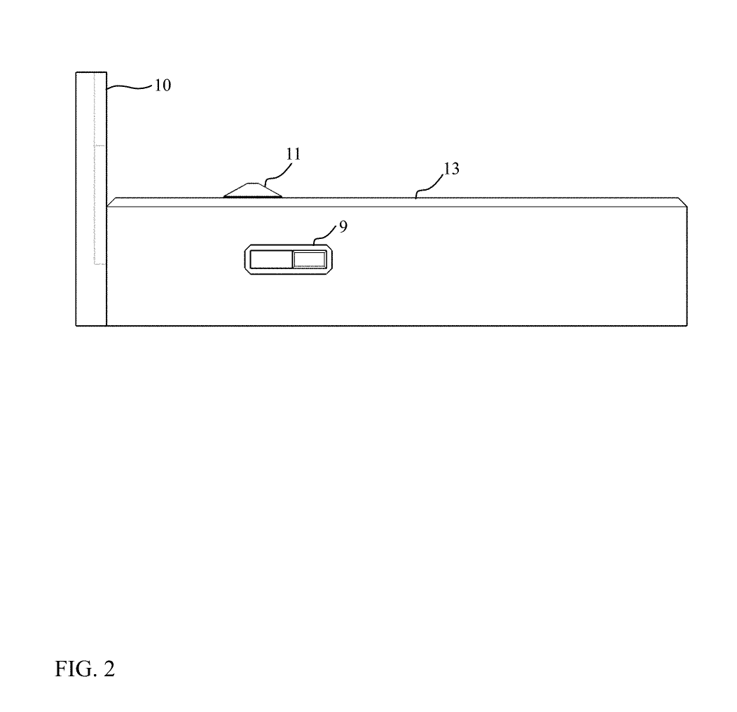

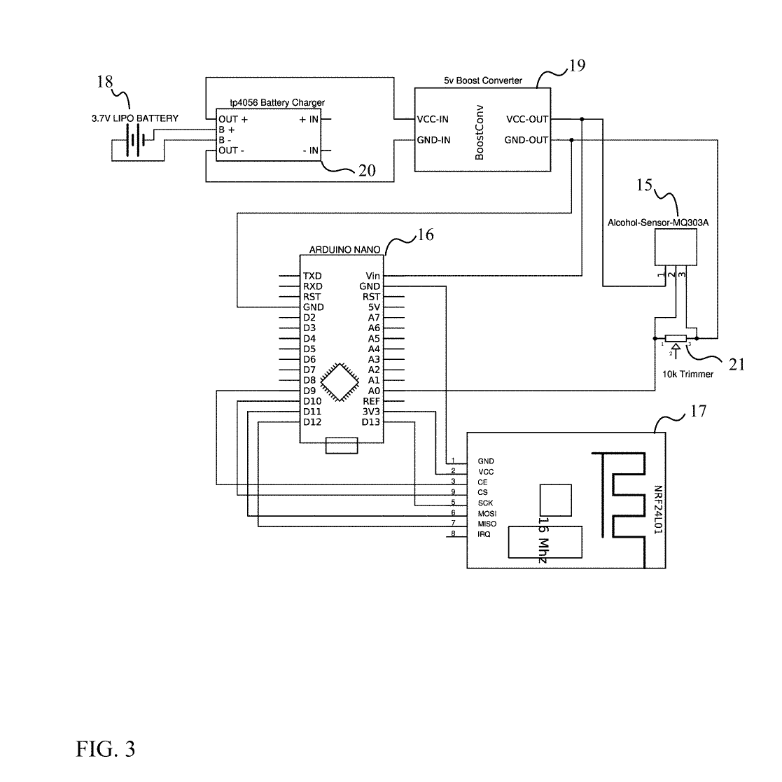

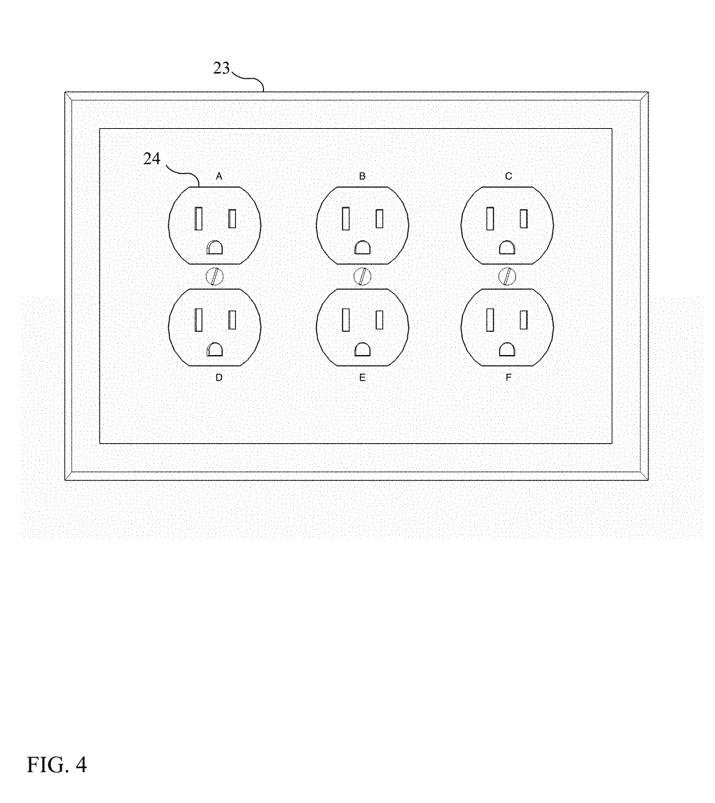

The first embodiment of the breath alcohol-measurement system includes a handheld, battery-powered, alcohol gas-sensing device, shown in FIGS. 1 and 2, that wirelessly transmits data to a receiving base unit shown in FIG. 4. The handheld device is comprised of an enclosure made of plastic 13 or a similar material, an alcohol gas sensor 15, a processor 16 to analyze the signal from the gas sensor, and a wireless transceiver 17 to send the processed data. An electrical schematic of the transmitter for this preferred embodiment is shown in FIG. 3. This embodiment is powered by a 3.7 volt lithium polymer battery (LiPo) 18 which is boosted to the 5 volt operating voltage of the micro controller 16 by a boost converter 19. The LiPo battery can be recharged with a 5 volt power supply through a charge protection circuit 20 to eliminate the risk of damaging the battery. In this embodiment the wireless transmitter is a 2.4 GHz radio transceiver 17, but any other wireless transmitter with similar capabilities could be used. A mouth piece 10 mounted on the enclosure directs the breath sample to the alcohol gas sensor 15. A user activates the handheld device by sliding a switch 9 located on the side of the device. Once activated, the gas sensor begins sampling the ambient air to calibrate while alerting the user of this action with a red light 11. After the calibration period is complete, a green light 12 prompts the user to begin blowing into the mouth piece of the device. The sensitivity of the alcohol gas sensor can also be manually set by adjusting a 10 kOhm trimmer potentiometer 21. The handheld device measures the alcohol content of the user's breath and wirelessly transmits the data to the base unit. After transmitting the data the microprocessor enters a sleep mode to preserve battery and can be awoken by pressing a push button 14 located on the side opposite the switch.

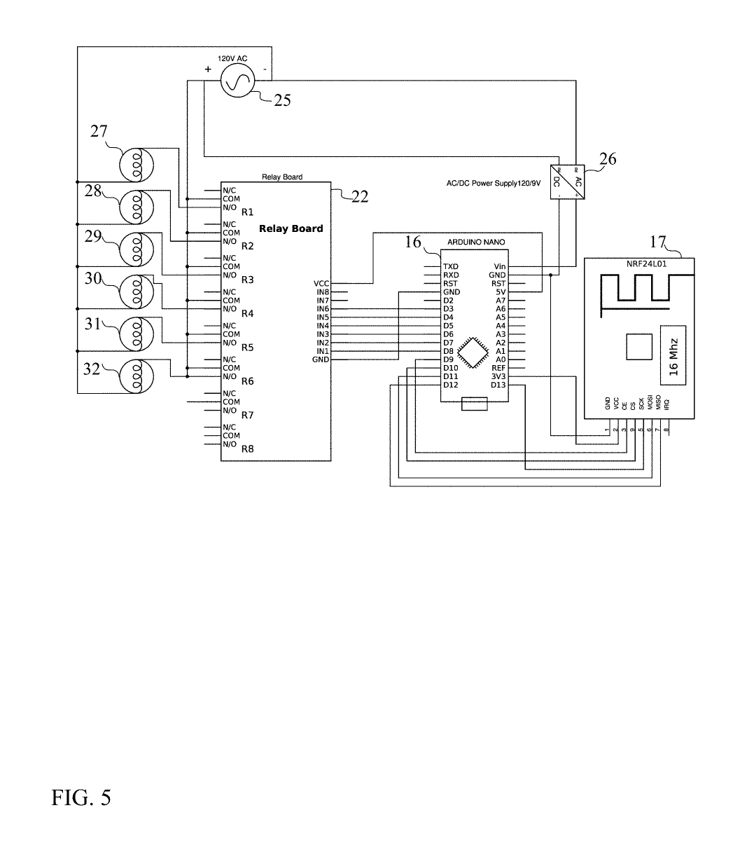

In this embodiment, the receiving unit, (front view shown in FIG. 4 and electrical schematic shown in FIG. 5) consists of a processor 16 and wireless transceiver 17 similar to the transmitting unit, eight electromechanical or solid-state relays 22, and an enclosure 23 made of plastic or a similar material containing the electronics just mentioned. Fitted into the top of the enclosure, there are six electrical power-outlet sockets 24 lettered A through F. The base unit is powered through a 110-220 volt 25 alternating current to 5 volt direct current transformer and rectifier circuit 26 that is plugged into a mains power supply wall socket. Each of the 6 electrical-outlet plugs on the top of the base unit is wired to a relay and mains power from the wall socket such that if the relay is in the closed position, the circuit is complete and mains power is provided to the corresponding outlet plug. The six relays are wired to output pins of the processor. FIG. 5 shows how the lights 27-32 are wired to the relays and the mains power source.

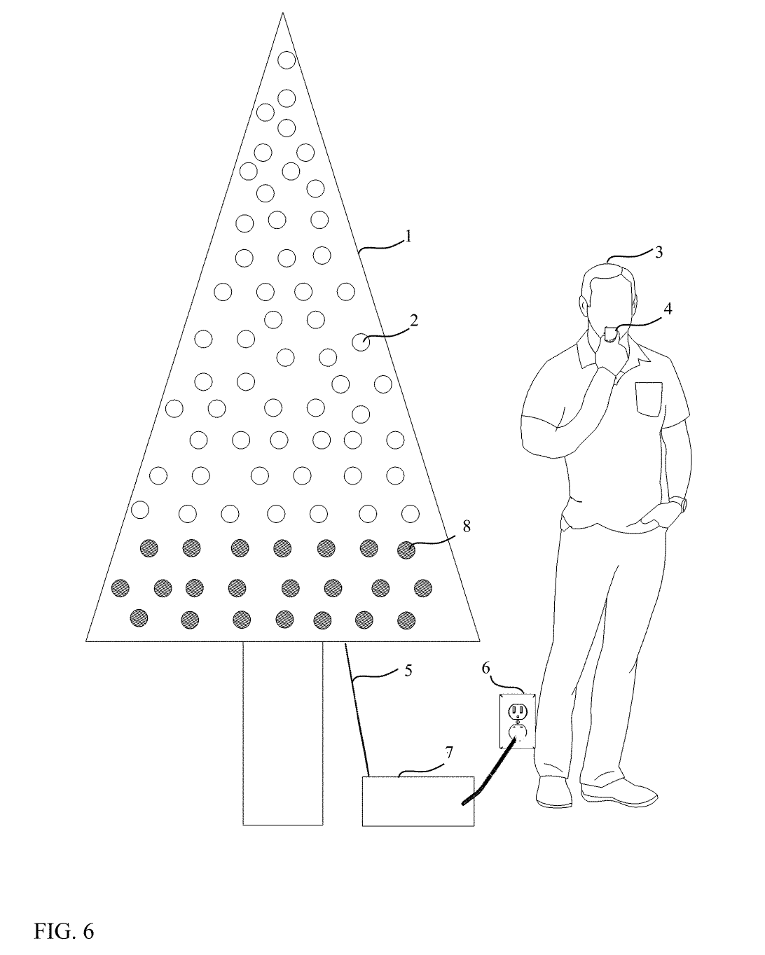

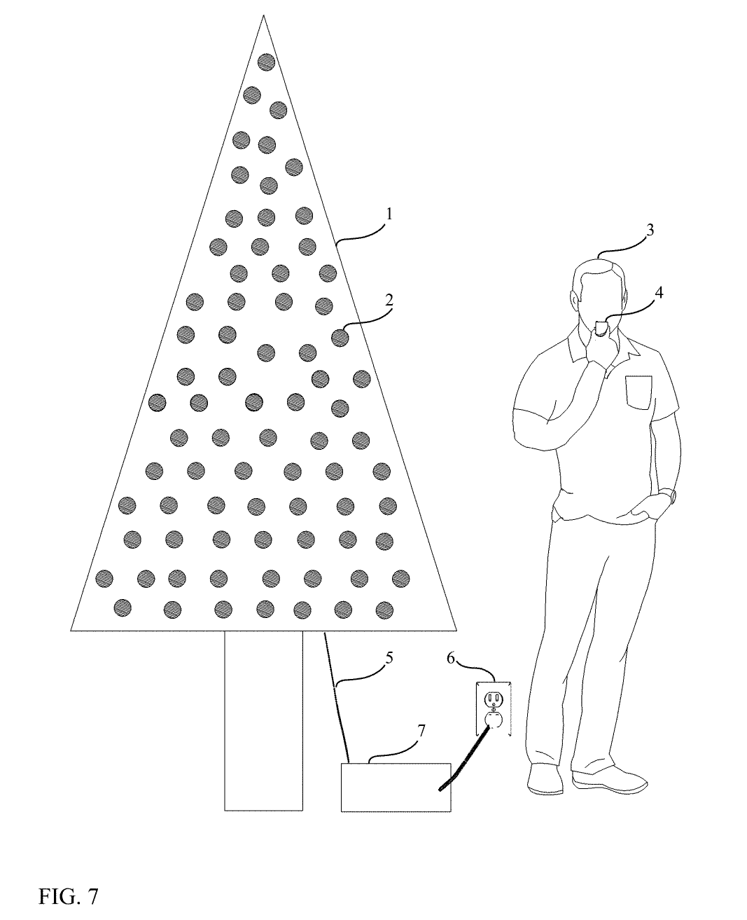

### OPERATION—FIGS. 6-7

FIGS. 6 and 7 illustrate the use of this preferred embodiment. A user 3 can plug up to six holiday string lights 5 into the six wall sockets on the top of the base unit 7. It is intended that the user wrap each of the six strings up a decorative tree 1 (such as a Christmas tree) in ascending order—the first strand wrapped around the base and the last strand wrapped around the top of the tree.

The user 3 blows into the transmitter unit 4. The data is sent wirelessly to the receiving base unit 7 which is powered through a 110-220 volt AC power outlet 6. The tree 1 has a series of string lights wrapped around it whose plugs are plugged into the base unit. The base unit receives the incoming breath alcohol data from the transmitter 4 and sends it to the processor. The data, determined by the concentration of alcohol sensed by the alcohol gas sensor, ranges from 0 to 70 with 0 meaning there was no alcohol sensed and 90 meaning the sensor is fully saturated. If the value is between 0 and 10, the processor would not activate any of the relays and the lights will remain unlit. If the value is between 11 and 20 the processor would activate the first relay corresponding to the first outlet socket. This scenario is illustrated in FIG. 6. The first string light corresponding to outlet A is illuminated 8 while the rest remain unlit 2. If the value is between 21 and 30 the processor would activate the first relay and the second relay corresponding to the outlets A and B. If the value is between 61 and 70 the processor would activate all six relays. Thus, the Christmas tree illuminates proportionally to an increasing alcohol concentration sensed.

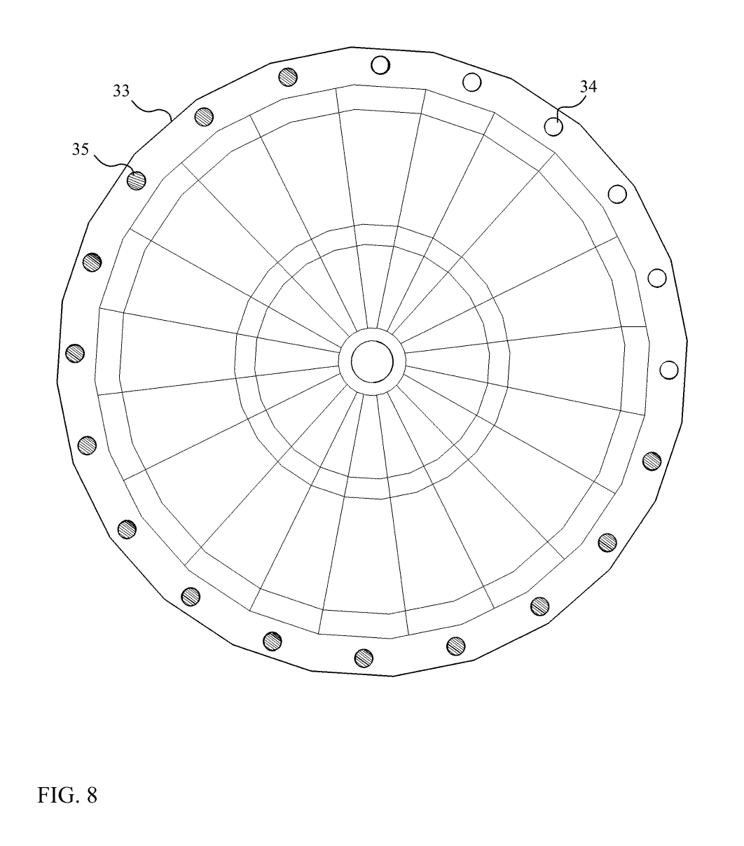

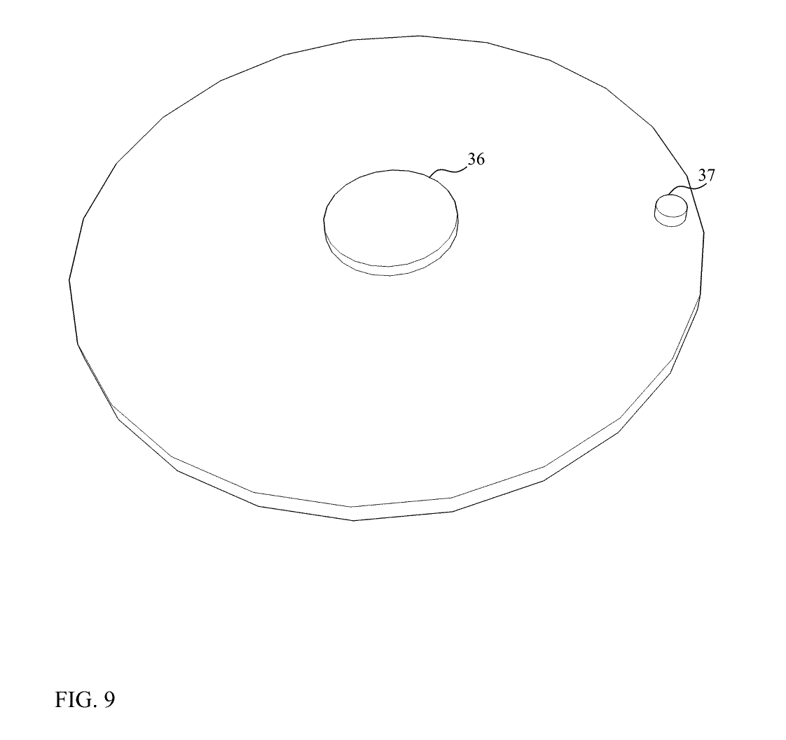

### DETAILED DESCRIPTION—FIGS. 8-9—SECOND EMBODIMENT

In a second embodiment of the present invention, with a similar transmitting unit as the first, the base unit electronics are embedded in an enclosure 36 located on the back side of a dart board 33. This receiving unit is powered by a rechargeable LiPo battery with charge protection circuit and booster converter circuit in a similar manner to the first embodiment. This allows the receiving dart board unit to be wireless so it can be hung in a wider variety of locations. On the back of the dart board there is a hanging point 37 that extends to the depth of the plastic enclosure to allow the board to be hung flush against a surface. An array of light emitting diodes 34 (LEDs) are positioned around the circumference of the dart board and are wired to the outputs of the processor. The outputs of the processor are wired to the gates of transistors that either (i) complete a circuit to provide 5V power from the boost converter to the LEDs when open, or (ii) disconnect the LEDs from said power source. In the default state, the LEDs are illuminated 35. When the processor receives a value from the transmitter, it will turn off the LEDs starting from the twelve o'clock position and proceeding in a clockwise manner proportionally to the concentration of alcohol sensed—the greater concentration of alcohol, the fewer LEDs illuminated. FIG. 9 shows the scenario where a minimal amount of alcohol has been sensed by the breathalyzer transmitter unit and thus only three LEDs are not illuminated.

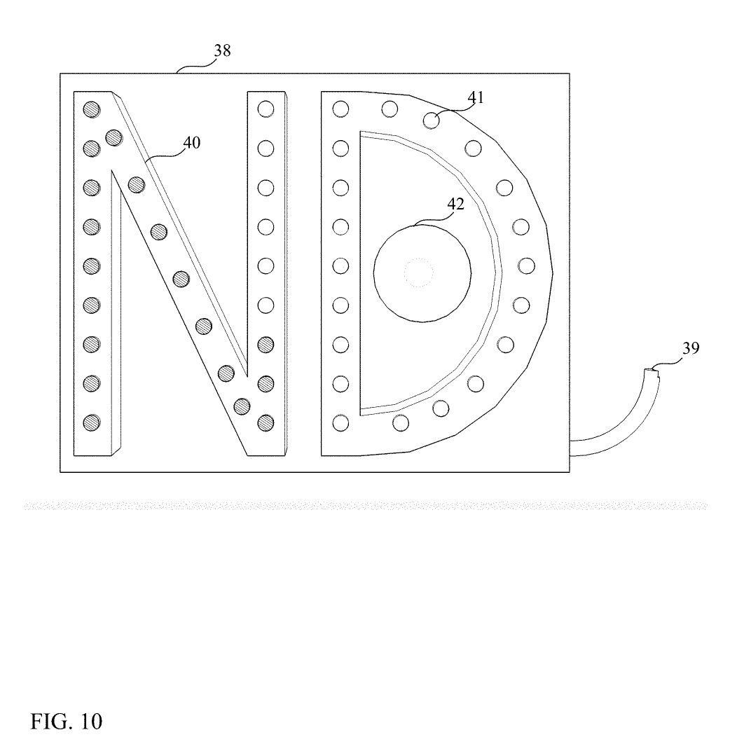

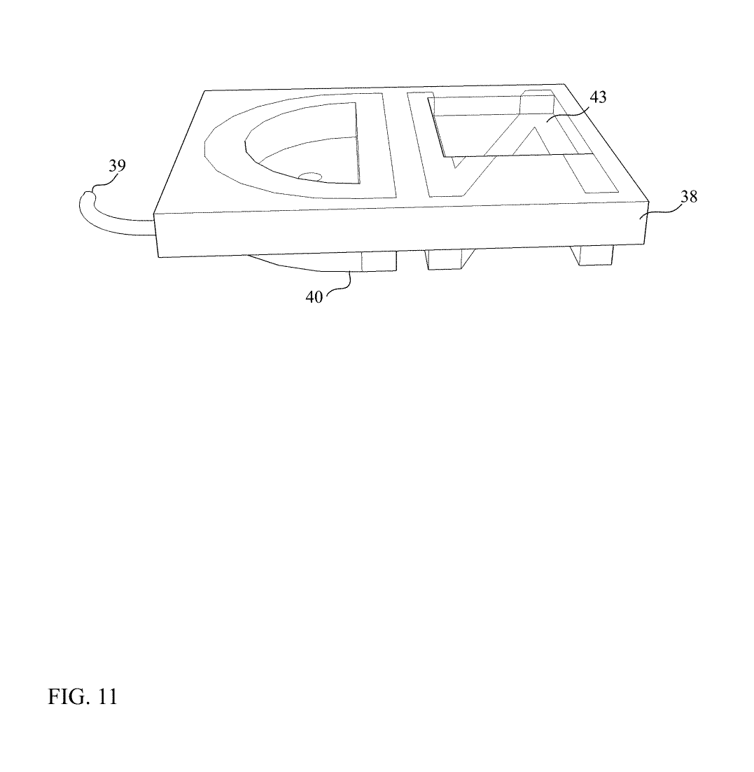

### DETAILED DESCRIPTION—FIGS. 10-11—THIRD EMBODIMENT

A third embodiment of the present invention, comprises a single unit which processes the breath alcohol data from a user and controls the sensory output. The sensory output in this embodiment consists of an array of LEDs, similar to the previous embodiment. It also a small speaker that is wired to the output of the processor. The alcohol gas sensor is directly wired to the analog input of the processor. As shown in FIG. 10, the sensory output is integrated with a sports team sign 38. The LEDs 41 cover the initials 40 of the sports team and the speaker 42 is positioned within the letter ‘D’ of the initials. The electronics are fitted into a cut out 43 on the back of the sign as shown in FIG. 11.

To activate the device, the user blows into the mouth piece 39 and into the alcohol gas sensor. The processor is awoken by the incoming breath alcohol data and begins flashing all LEDs on the sign in 500 millisecond intervals. The alcohol gas sensor is also sensitive to changes in temperature and humidity so it is capable of being activated by a user with no alcohol on their breath. Once the processor is awoken, it receives the alcohol gas data for 8 seconds, turns off all the LEDs, and plays two 1,000 Hz tones for 250 milliseconds with a 250 millisecond pause in between to alert the user to stop blowing. The full range of alcohol concentration that can be detected by the gas sensor is divided into 8 steps. For the minimum threshold of alcohol concentration, the processor illuminates the first set of LEDs starting at the bottom left hand corner of the ‘N’ while simultaneously playing a sweeping tone on the speaker from 100 Hz to 350 Hz over the span of 250 milliseconds. If the processor senses the second step of increasing alcohol concentration, the first two sets of LEDs illuminate on the ‘N’ sequentially over the span of 500 milliseconds while the speaker plays a tone from 100 Hz to 600 Hz. For every further step of increasing alcohol concentration, another set of LEDs illuminate one after another over a span of time equal to the number of steps multiplied by 250 milliseconds. At the same time the speaker plays a sweeping tone starting at 100 Hz to 2,100 Hz depending on the concentration sensed. When the alcohol concentration sensed is high enough that the ‘N’ is fully illuminated, the LEDs begin illuminating the ‘D’ in a clockwise fashion, starting at the top left corner. In this way, the device utilizes both visual and auditory sensory outputs to qualitatively represent breath alcohol concentration.

What is claimed is:

1. An apparatus for qualitatively representing breath alcohol concentration, the apparatus comprising:
an electronic device capable of determining the breath alcohol concentration from a breath sample blown into the device;
a sensory output means for qualitatively representing said alcohol concentration;
a decorative accessory with which said sensory output is integrated

2. The apparatus of claim 1 wherein the sensory output further comprises:
an array of lights, a noise generating device, or a vibrational apparatus whereby the visual, auditory or vibrational properties of said sensory output are determined by the measured alcohol concentration

3. The apparatus of claim 1 wherein the decorative accessory further comprises:
ornamentation associated with holidays, sporting events or other festive occasions.
